(19) United States
(12) Patent Application Publication
(10) Pub. No.: US 2005/0221507 A1
(43) Pub. Date: Oct. 6, 2005

(54) Method to detect molecular binding by surface-enhanced Raman spectroscopy

(75) Inventors: Tae-Woong Koo, Cupertino, CA (US); Selena Chan, San Jose, CA (US)

Correspondence Address: DLA PIPER RUDNICK GRAY CARY US, LLP, 4365 EXECUTIVE DRIVE, SUITE 1100, SAN DIEGO, CA, 92121-2133, US

(73) Assignee: Intel Corporation, Santa Clara, CA (US)

(21) Appl. No.: 10/814,981

(22) Filed: Mar. 30, 2004

(51) Int. Cl. G01N 33/553

(52) U.S. Cl. 436/525

(57) ABSTRACT

Provided herein are methods for detecting molecular binding using surface enhanced Raman spectroscopy (SERS). The SERS signal can be generated by associating one of the binding partners with a SERS-active particle or substrate. Binding is detected by detecting a change in a SERS signal after two binding partners are contacted with each other as compared to before the binding partners are contacted with each other. The method is useful for detecting binding of biomolecules such as antibodies to antigens and receptors to ligands.

### BACKGROUND OF THE INVENTION

1. Field of the Invention

The invention relates generally to detection and analysis of biomolecules, and more specifically to detection of binding between biomolecules.

2. Background Information

Molecular binding assays, such as immunoassays, are frequently used for the detection of analytes in serum, plasma, urine or other body fluid samples for medical and diagnostic purposes. A plethora of analytes are detectable by molecular binding assays. However, for many analytes the requirements for analytical and functional sensitivity are becoming more and more demanding.

Conventionally, molecular binding is observed by detecting fluorescent or radioactive labels on either an antibody or a target molecule. However, conventional methods are often affected by strong background signal due to non-specific binding, which generates misleading information about the binding events or limits the functional sensitivity of the method. For example, non-specific binding of an antibody or contamination from residual dye molecules, generate erroneously high background signal. Non-specific binding is inherent in traditional assay methods and difficult to avoid completely.

### BRIEF DESCRIPTION OF THE DRAWINGS

FIG. 1 illustrates signal level change during a method provided herein.

FIG. 2 schematically illustrates an exemplary method provided herein.

FIGS. 3A and 3B provide a SERS signature of an antibody molecule (FIG. 3A) and a negative control signal generated in the absence of the antibody (FIG. 3B).

### DETAILED DESCRIPTION OF THE INVENTION

The methods disclosed herein are useful for detecting biomolecular binding between a first specific binding pair member and a second specific binding pair member by detecting a change in the surface enhanced Raman spectroscopy (SERS) signal generated by the first specific binding pair member upon binding to the second specific binding pair member. The methods disclosed herein do not require the labeling process of traditional fluorescent assays, such as immunoassays, used for detecting binding of a first biomolecule to a second biomolecule. Since labels are not used and/or fluorescent detection is not employed, the background signal of an assay is greatly reduced. Furthermore, modification of a biomolecule, such as binding a label to a biomolecule, which is difficult and can interfere with the structure and/or activity of the biomolecule, is not necessary. Therefore, by using SERS binding events can be detected without using fluorescence labels, resulting in an increased sensitivity and increased accuracy.

The methods provided herein are based in part on the fact that certain biomolecules are known to generate strong SERS signals. Furthermore, SERS signals are sensitive to chemical and environmental changes. (Efrima S. and Bronk B. V., (1998), Journal of Physical Chemistry B, 102:5947; Lee N S, Hsieh Y Z, Paisley R. F., and Morris M. D. (1988), Anal. Chem. 64:442; Wood E, Sutton C, Beezer A E, Creighton J A, Davis A F and Mitchell J. C., (1997) International Journal of Pharmaceutics, 154:115). Finally, binding events between members of many different types of specific binding pairs, such as antibody and antigen, and many different specific binding pairs, are known.

Accordingly, in one embodiment, provided herein is a method to detect binding of a first specific binding pair member to a second specific binding pair member. The method includes associating the first specific binding pair member with a surface-enhanced Raman scattering (SERS)-active particle or substrate, contacting the first specific binding pair member associated with the SERS-active particle or substrate with a second specific binding pair member, and detecting binding of the second specific binding pair member to the first specific binding pair member. The binding is typically determined by detecting a difference in a SERS signal generated by the first specific binding pair member before contacting the first specific binding pair member with the second specific binding pair member, as compared with a SERS signal generated after contacting the first specific binding pair member with the second specific binding pair member. By detecting binding of a first specific binding pair member to a second specific binding pair member, methods disclosed herein allow detection of molecular interactions between a first molecule and a second molecule. Methods described herein can be used to detect interaction between virtually any molecules provided that one of the molecules generates a detectable SERS signal when associated with a SERS-active particle or substrate, and this SERS signal is affected by binding of the first molecule to the second molecule. For example, in one aspect, the first specific binding pair member is an antibody and the second specific binding pair member is an antigen that is specifically bound by the antibody. In another aspect, a first specific binding pair member is a receptor and a second specific binding pair member is a ligand.

As used herein, “a” or “an” can mean one or more than one of an item.

As used herein, the terms “analyte” refer to any atom, chemical, molecule, compound, composition or aggregate of interest for detection and/or identification. Non-limiting examples of analytes include an amino acid, peptide, polypeptide, protein, glycoprotein, lipoprotein, nucleoside, nucleotide, oligonucleotide, nucleic acid, sugar, carbohydrate, oligosaccharide, polysaccharide, fatty acid, lipid, hormone, metabolite, cytokine, chemokine, receptor, neurotransmitter, antigen, allergen, antibody, substrate, metabolite, cofactor, inhibitor, drug, pharmaceutical, nutrient, prion, toxin, poison, explosive, pesticide, chemical warfare agent, biohazardous agent, radioisotope, vitamin, heterocyclic aromatic compound, carcinogen, mutagen, narcotic, amphetamine, barbiturate, hallucinogen, waste product and/or contaminant.

A “biological sample” includes, for example, urine, blood, plasma, serum, saliva, semen, stool, sputum, cerebral spinal fluid, tears, mucus, and the like. In certain aspects, the biological sample is from a mammalian subject, for example a human subject. The biological sample can be virtually any biological sample, as long as the sample contains or may contain a second specific binding pair member. For example, the sample can be suspected of containing a protein that has an epitope recognized by an antibody included as the first specific binding pair member. The biological sample can be a tissue sample which contains, for example, 1 to 10,000,000; 1000 to 10,000,000; or 1,000,000 to 10,000,000 somatic cells. The sample need not contain intact cells, as long as it contains sufficient quantity of a specific binding pair member for the methods provided herein. According to aspects of the methods provided herein, wherein the biological sample is from a mammalian subject, the biological or tissue sample can be from any tissue. For example, the tissue can be obtained by surgery, biopsy, swab, stool, or other collection method. In other aspects, the biological sample contains, or is suspected to contain, or at risk for containing, a pathogen, for example a virus or a bacterial pathogen.

As used herein, the term “nanocrystalline silicon” refers to silicon that comprises nanometer-scale silicon crystals, typically in the size range from 1 to 100 nanometers (nm). “Porous silicon” refers to silicon that has been etched or otherwise treated to form a porous structure.

As used herein, “operably coupled” means that there is a functional interaction between two or more units of an apparatus and/or system. For example, a Raman detector may be “operably coupled” to a computer if the computer can obtain, process, store and/or transmit data on Raman signals detected by the detector.

The term “binds specifically” or “specific binding activity,” when used in reference to an antibody means that an interaction of the antibody and a particular epitope has a dissociation constant of at least about 1×10⁻⁶, generally at least about 1×10⁻⁷, usually at least about 1×10⁻⁸, and particularly at least about 1×10⁻⁹ or 1×10⁻¹⁰ or less.

As used herein, the term “antibody” is used in its broadest sense to include polyclonal and monoclonal antibodies, as well as antigen binding fragments of such antibodies. The invention includes whole antibodies and functional fragments thereof. The term antibody as used in this invention is meant to include intact molecules as well as fragments thereof, such as Fab and F(ab′)₂, Fv and SCA fragments which are capable of binding an epitopic determinant.
(1) An Fab fragment consists of a monovalent antigen-binding fragment of an antibody molecule, and can be produced by digestion of a whole antibody molecule with the enzyme papain, to yield a fragment consisting of an intact light chain and a portion of a heavy chain.
(2) An Fab′ fragment of an antibody molecule can be obtained by treating a whole antibody molecule with pepsin, followed by reduction, to yield a molecule consisting of an intact light chain and a portion of a heavy chain. Two Fab′ fragments are obtained per antibody molecule treated in this manner.
(3) An (Fab′)₂ fragment of an antibody can be obtained by treating a whole antibody molecule with the enzyme pepsin, without subsequent reduction. A (Fab′)₂ fragment is a dimer of two Fab′ fragments, held together by two disulfide bonds.
(4) An Fv fragment is defined as a genetically engineered fragment containing the variable region of a light chain and the variable region of a heavy chain expressed as two chains.
(5) A single chain antibody (“SCA”) is a genetically engineered single chain molecule containing the variable region of a light chain and the variable region of a heavy chain, linked by a suitable, flexible polypeptide linker.

The term “antibody” as used herein includes naturally occurring antibodies as well as non-naturally occurring antibodies, including, for example, single chain antibodies, chimeric, bifunctional and humanized antibodies, as well as antigen-binding fragments thereof. Such non-naturally occurring antibodies can be constructed using solid phase peptide synthesis, can be produced recombinantly or can be obtained, for example, by screening combinatorial libraries consisting of variable heavy chains and variable light chains (see Huse et al., Science 246:1275-1281 (1989)). These and other methods of making, for example, chimeric, humanized, CDR-grafted, single chain, and bifunctional antibodies are well known to those skilled in the art (Winter and Harris, Immunol. Today 14:243-246, 1993; Ward et al., Nature 341:544-546, 1989; Harlow and Lane, Antibodies: A laboratory manual (Cold Spring Harbor Laboratory Press, 1988); Hilyard et al., Protein Engineering: A practical approach (IRL Press 1992); Borrabeck, Antibody Engineering, 2d ed. (Oxford University Press 1995)).

Methods for raising polyclonal antibodies, for example, in a rabbit, goat, mouse or other mammal, are well known in the art (see, for example, Green et al., “Production of Polyclonal Antisera,” in Immunochemical Protocols (Manson, ed., Humana Press 1992), pages 1-5; Coligan et al., “Production of Polyclonal Antisera in Rabbits, Rats, Mice and Hamsters,” in Curr. Protocols Immunol. (1992), section 2.4.1). In addition, monoclonal antibodies can be obtained using methods that are well known and routine in the art (Harlow and Lane, supra, 1988).

As used in this invention, the term “epitope” refers to an antigenic determinant on an antigen, to which the paratope of an antibody binds. Antigenic determinants usually consist of chemically active surface groupings of molecules, such as amino acids or sugar side chains, and can have specific three-dimensional structural characteristics, as well as specific charge characteristics.

Examples of types of immunoassays of the invention include competitive and non-competitive immunoassays in either a direct or indirect format. Those of skill in the art will know, or can readily discern, other immunoassay formats without undue experimentation.

In performing a method of the present invention, “blocking agents” can be included in an incubation medium. “Blocking agents” are added to minimize non-specific binding to a surface and between molecules.

“Nucleic acid” means DNA, RNA, single-stranded, double-stranded or triple stranded and any chemical modifications thereof. Virtually any modification of the nucleic acid is contemplated. A “nucleic acid” can be of almost any length, from 10, 20, 30, 40, 50, 60, 75, 100, 125, 150, 175, 200, 225, 250, 275, 300, 400, 500, 600, 700, 800, 900, 1000, 1500, 2000, 2500, 3000, 3500, 4000, 4500, 5000, 6000, 7000, 8000, 9000, 10,000, 15,000, 20,000, 30,000, 40,000, 50,000, 75,000, 100,000, 150,000, 200,000, 500,000, 1,000,000, 1,500,000, 2,000,000, 5,000,000 or even more bases in length, up to a full-length chromosomal DNA molecule.

The term “receptor” is used to mean a protein, or fragment thereof, or group of associated proteins that selectively bind a specific substance called a ligand. Upon binding its ligand, the receptor triggers a specific response in a cell.

The term “polypeptide” is used broadly herein to mean two or more amino acids linked by a peptide bond. The term “fragment” or “proteolytic fragment” also is used herein to refer to a product that can be produced by a proteolytic reaction on a polypeptide, i.e., a peptide produced upon cleavage of a peptide bond in the polypeptide. A polypeptide of the invention contains at least about six amino acids, usually contains about ten amino acids, and can contain fifteen or more amino acids, particularly twenty or more amino acids. It should be recognized that the term “polypeptide” is not used herein to suggest a particular size or number of amino acids comprising the molecule, and that a peptide of the invention can contain up to several amino acid residues or more. A protein is a polypeptide that includes other chemical moieties other than amino acids, such as phosphate groups or carbohydrate moiety.

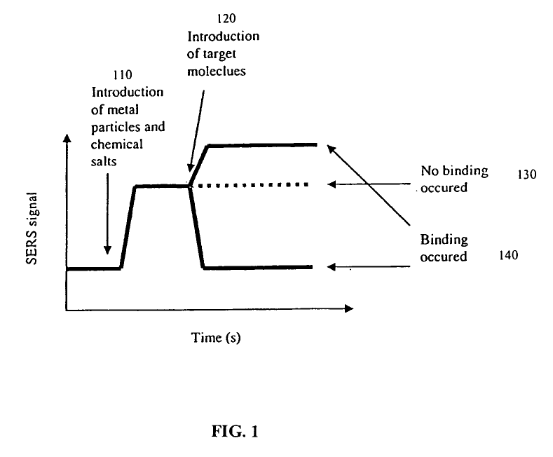

FIG. 1 provides a hypothetical graph which illustrates expected SERS signal level changes during various steps of methods provided herein. Before association of the first specific binding pair member with a SERS-active substrate or particle, little or no SERS effect is observed, and the Raman signal of the first specific binding pair member, such as an antibody, in certain aspects of the invention, is weak. The SERS signal generated by a first specific binding pair member is strengthened by associating the first specific binding pair member with a SERS-active particle or substrate, for example by adsorbing the first specific binding pair member to a metal particle, after introduction of metal particles and optionally, chemical salts 110. Typically after the association, which in certain aspects of the methods disclosed herein is detected by an increase in SERS signal, a second specific binding pair member, sometimes referred to herein as a target molecule, is introduced 120 to contact and bind the first specific binding pair member. As a result of the binding of the first specific binding pair member to the second specific binding pair member, the SERS signal is changed, thus allowing detection of the binding. For example, an increase in signal or a decrease in signal indicates that binding has occurred 140. No change in the SERS signal 130 after contacting the first specific binding pair member with the second specific binding pair member, indicates that binding has not occurred, thus indicating the absence of a detectable level of the target molecule 130. Therefore, by monitoring a SERS signal before and after the first specific binding pair member is contacted with the second specific binding pair member, the binding of the first specific binding pair member to the second specific binding pair member can be detected.

The difference in the SERS signal of the first specific binding pair member before versus after contact with the second specific binding pair member is the result of a disruption or enhancement of SERS signal generation by the molecular binding events of the first specific binding pair member and the second specific binding pair member. While not wanting to be bound by a particular theory, it is believed that in certain examples, the SERS signal generated by the first specific binding pair member is reduced when a second specific binding pair member quenches the SERS signal of the first specific binding pair member. This quenching of the SERS signal can be the result, for example, of dissociation of the first specific binding pair member from the SERS-active substrate upon binding of the second specific binding pair member to the first specific binding pair member. In other words, the binding force of the first specific binding pair member to the second specific binding pair member can be stronger than the forces associating the first specific binding pair member to the SERS-active metal particle or surface and can dissociate the first specific binding pair member from the SERS-active particle or substrate.

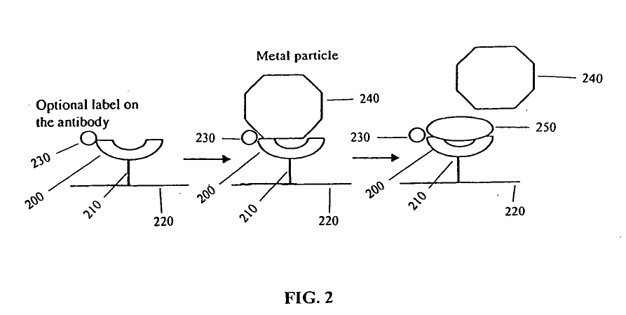

FIG. 2 illustrates a specific example of a method for detecting binding of a first specific binding pair member to a second specific binding pair member according to a method disclosed herein. A first specific binding pair member 200 is immobilized on a solid support 220 using an immobilization group (e.g. a crosslinking agent) 210. A SERS-active particle or substrate, for example a metal particle 240, is associated with the first specific binding pair member 200. Binding of the first specific binding pair member 200 to the second specific binding pair member 250 can then dissociate the metal particle 240 from the first specific binding pair member 200, resulting in a reduced SERS signal.

The first specific binding pair member 200 can be associated with a label 230 to enhance the SERS signal of the first specific binding pair member 200. In other examples, the first specific binding pair member 200 can generate a SERS signal of its own when positioned close to the SERS-active particle or surface. In another aspect of the invention, binding of a first specific binding pair member 200 to a second specific binding pair member 250 results in an increase in the SERS signal. For example, a second specific binding pair 250 can bring the first specific binding pair member 200 into closer proximity to the SERS active particle or surface 220, thereby increasing the SERS signal. Alternatively, the SERS signal can be generated by the second specific binding pair member 250 or by both the first specific binding pair member 200 and the second specific binding pair member 250.

As indicated above, to increase the SERS signal generated by the first specific binding pair member in the methods disclosed herein, the first specific binding pair member is typically associated with a SERS-active particle or substrate, such as a metal particle. Accordingly, detection of the first specific binding pair member is accomplished using SERS. Typically, when a first specific binding pair member is associated with a SERS-active particle or substrate it is adjacent to the SERS-active particle or substrate and brought to within 10 nm to 50 nm of the SERS-active particle or substrate (Chang and Furtak, Surface-enhanced Raman scattering, Plenum Press (1982)).

Certain embodiments of the invention include the use of SERS-active particles to enhance the Raman signal obtained from the first specific binding pair member. For example, metal particles can be associated with specific binding pair members in the methods herein. The metal particles are typically colloidal silver, gold, copper, platinum, or other metallic particles of specific size and shape, which generate strong plasmon resonance.

In some aspects, the metal particles are nanoparticles that contain a SERS-active metal, such as silver or gold nanoparticles. Any nanoparticle capable of providing a surface enhanced Raman spectroscopy (SERS) signal may be used. In alternative embodiments of the invention, the nanoparticles can be nanoprisms (Jin et al., Science 294:1902-3 (2001)). In various aspects, nanoparticles of between 1 nm and 2 micrometers (μm) in diameter can be used. In alternative aspects, nanoparticles of between 2 nm to 1 μm, 5 nm to 500 mm, 10 run to 200 nm, 20 nm to 100 mm, 30 nm to 80 mm, 40 nm to 70 nm or 50 to 60 nm diameter are contemplated. In certain embodiments of the invention, nanoparticles with an average diameter of 5 to 200 nm, 10 to 50 mm, 50 to 100 nm or about 100 nm are contemplated. The nanoparticles may be approximately spherical, rod-like, edgy, faceted or pointy in shape, although nanoparticles of any shape or of irregular shape may be used. Methods of preparing nanoparticles are known (e.g., U.S. Pat. Nos. 6,054,495; 6,127,120; 6,149,868; Lee and Meisel, J. Phys. Chem. 86:3391-3395, 1982; Jin et al., 2001). Nanoparticles may also be obtained from commercial sources (e.g., Nanoprobes Inc., Yaphank, N.Y.; Polysciences, Inc., Warrington, Pa.).

The nanoparticles can be single nanoparticles and/or aggregates of nanoparticles (e.g. colloidal nanoparticles). The nanoparticles can be cross-linked to produce aggregates of nanoparticles, such as dimers, trimers, tetramers or other aggregates. Heterogeneous mixtures of aggregates of different size, or homogenous populations of nanoparticles can also be used. Aggregates containing a selected number of nanoparticles (e.g., dimers, trimers, etc.) can be enriched or purified by known techniques, such as ultracentrifugation in sucrose solutions. Nanoparticle aggregates of about 100, 200, 300, 400, 500, 600, 700, 800, 900 to 1000 nm in size or larger are contemplated.

Methods of cross-linking nanoparticles are known (e.g., Feldheim, “Assembly of metal nanoparticle arrays using molecular bridges,” The Electrochemical Society Interface, Fall, 2001, pp. 22-25). Gold nanoparticles can be cross-linked, for example, using bifunctional linker compounds bearing terminal thiol or sulfhydryl groups. Upon reaction with gold nanoparticles, the linker forms nanoparticle dimers that are separated by the length of the linker. Linkers with three, four or more thiol groups may be used to simultaneously attach to multiple nanoparticles (Feldheim, 2001). The use of an excess of nanoparticles to linker compounds prevents formation of multiple cross-links and nanoparticle precipitation. Aggregates of silver nanoparticles can be formed by standard synthesis methods known in the art.

The nanoparticles can be modified to contain various reactive groups before they are attached to linker compounds. Modified nanoparticles are commercially available, such as Nanogold® nanoparticles from Nanoprobes, Inc. (Yaphank, N.Y.). Nanogold® nanoparticles may be obtained with either single or multiple maleimide, amine or other groups attached per nanoparticle. The Nanogold® nanoparticles are also available in either positively or negatively charged form. Such modified nanoparticles may be attached to a variety of known linker compounds to provide dimers, trimers or other aggregates of nanoparticles.

The type of linker compound used is not limiting, so long as it results in the production of small aggregates of nanoparticles that will not simultaneously precipitate in solution. The linker group may include phenylacetylene polymers (Feldheim, 2001). Alternatively, linker groups may comprise polytetrafluoroethylene, polyvinyl pyrrolidone, polystyrene, polypropylene, polyacrylamide, polyethylene or other known polymers. The linker compounds of use are not limited to polymers, but may also include other types of molecules such as silanes, alkanes, derivatized silanes or derivatized alkanes.

The first specific binding pair member can optionally be immobilized on an immobilization substrate before it is associated with the SERS-active surface. This immobilization facilitates methods according to certain aspects of the methods disclosed herein, for example by making it easier to separate a disassociated SERS-active surface from a first binding pair member upon binding of a second binding pair member. Methods are known in the art for immobilizing various molecules that can act as specific binding pair members, as discussed in further detail herein.

Methods provided herein can be used to detect molecular interaction (i.e. binding) of virtually any specific binding pair member. As used herein, the term “specific binding pair member” refers to a molecule that specifically binds or selectively hybridizes to, or interacts with, another member of a specific binding pair. Specific binding pair members include, for example a receptor and a ligand, or an antigen and an antibody. An “analyte,” which can be one of the specific binding pair members, includes, but is not limited to, a nucleic acid, a protein or peptide, a lipid, or a polysaccharide. For example, the first specific binding pair member can be a protein, such as an antibody molecule, or fragment thereof, and the second specific binding pair member can be a biomolecule, such as a protein, that includes an epitope recognized by the antibody. In one example, the first specific binding pair member is a receptor and the second specific binding pair member is a ligand.

In another example, the first or second specific binding pair member is a nucleic acid molecule that interacts with the first or second specific binding pair member. Accordingly, this embodiment can be used to identify nucleic acid molecules that interact with proteins, or proteins that interact with nucleic acid molecules. Since nucleic acid molecules provide a relatively strong SERS signal, in certain aspects wherein the second specific binding pair member is a nucleic acid, instead of detecting a change in a SERS signature of the first specific binding pair member, binding of a nucleic acid to the first specific binding pair member can be detected by detecting a SERS signal generated by the nucleic acid upon binding to the first specific binding pair member.

The use of a first and second specific binding pair member is illustrative. However, there can be additional specific binding pair members. For example, a third specific binding pair member that binds to the first specific binding pair member can be included. Binding of the second specific binding pair member can displace the third specific binding pair member in a competitive manner and, as a result, change the SERS signal generated by the first specific binding pair member.

The first specific binding pair member, such as an antibody, generally generates a strong SERS signal by itself when associated with a SERS-active surface, such as a metal particle. However, in certain aspects, in order to increase the SERS signal generated by the first specific binding pair member, the first specific binding pair member is associated with a SERS label. Alternatively, to enhance a SERS signature of a first specific binding pair member, the structure of the first specific binding pair member can be modified. For example, groups can be added to the first specific binding pair member, that increase a SERS signal. Such groups include, for example, nitrogen-containing groups such as amine groups, groups that include a double bond, and groups that include a ring structure, such as a benzene ring.

Methods are well known for attaching labels to many different biomolecules, such as antibodies. In certain aspects, the label is a nucleotide, or any other molecule which yields a strong SERS signal, as disclosed in further detail herein. For example, the SERS label can be deoxy-adenosine monophosphate. A dye can also be used to label the biomolecule, although care should be taken so that background signals remain at acceptable levels.

For example, the first specific binding pair member is associated with the SERS-active particle by immobilizing the first specific binding pair member on a standard substrate (e.g. glass or gold) and introducing SERS-active metal particles. In another embodiment, the first specific binding pair member is associated with the SERS-active substrate by immobilizing the first specific binding pair member on the SERS-active substrate, such as a porous silicon substrate that includes impregnated metals. As discussed herein, a SERS signal of the first specific binding pair member or its label is generated under laser excitation, typically after the first specific binding pair member is associated with the SERS-active particle or substrate. Further enhancement of the SERS signal can be obtained by using a non-standard SERS detection method such as SECARS.

In another embodiment, the invention provides a method to detect binding of an antibody, or fragment thereof, to an antigen, including immobilizing an antibody on an immobilization substrate, contacting the immobilized antibody with a metal particle to adsorb the immobilized antibody on the metal particle, contacting the immobilized antibody with an antigen, and detecting binding of the antigen to the antibody, or fragment thereof. Binding is detected by detecting a difference in a SERS signal generated by the antibody before versus after contacting the antibody with the antigen.

In another embodiment, the invention provides is a method to detect an analyte in a biological sample, including immobilizing a first specific binding pair member on a surface, contacting the immobilized first specific binding pair member with a metal particle to adsorb the immobilized first specific binding pair member on the metal particle, contacting the immobilized first specific binding pair member adsorbed on the metal particle with the biological sample; and detecting a surface-enhanced Raman scattering (SERS) signal generated by the immobilized first specific binding pair member before and after contacting the immobilized first specific binding pair member with the second specific binding pair member. A difference in the detected SERS signals is indicative of the presence of the analyte in the biological sample. In certain aspects, for example, the first specific binding pair member is an antibody, or fragment thereof. For example, the first specific binding pair member can be an antibody that binds the analyte.

In another embodiment, the invention provides a method to detect an antibody or a fragment thereof, including immobilizing an antibody, or fragment thereof, on a surface, contacting the antibody, or fragment thereof, with a metal particle to adsorb the immobilized antibody on the metal particle, and detecting a surface-enhanced Raman scattering (SERS) signal of the immobilized antibody, or fragment thereof, thereby detecting the antibody, or fragment thereof.

In certain aspects, the first specific binding pair member is associated with the SERS-active particle by mixing the first specific binding pair member with metal particles. In other aspects, the first specific binding pair member is associated with the SERS-active substrate by immobilizing the first specific binding pair member on the SERS-active substrate, such as a porous silicon substrate, that includes impregnated metals.

The reaction time for various steps of the methods provided herein is sufficient to allow contact of molecules included in the steps. For example, a reaction time for association of the first specific binding pair member to the SERS-active particle or substrate is sufficient to allow for the first specific binding pair member to bind the SERS-active particle or substrate. The rate at which the various reactants bind each other, and thus a minimum incubation time, is affected by a number of factors. These factors include the concentration of the reactants, the speed at which reactants are moved through a reaction chamber, and the size and shape of the reaction chamber, for example. Any of these factors can be altered in order to assure that an incubation time is sufficient to allow contact of the reactants. Reaction times for methods provided herein can range from 1 milliseconds to 1 hour, but typically ranges from 100 milliseconds to 60 minutes. For example, in certain aspects the incubation time is 100 milliseconds; 1, 2, 3, 4, 5, 10, 15, 20, 30, 45, or 60 seconds; or 2, 3, 4, 5, 6, 7, 8, 9, or 10, 20, 30, 45 or 60 minutes.

Several steps of methods provided herein utilize the association of a first specific binding pair member to a solid structure, such as a SERS-active particle or substrate, or an immobilization substrate. As indicated above, to facilitate performance of the methods disclosed herein, the first specific binding pair member can be immobilized to an immobilization substrate. Furthermore, the first specific binding pair member can be associated with a surface enhanced SERS-active particle or substrate. In certain aspects, the SERS-active particle or substrate is also the immobilization substrate. Methods are known for associating specific binding pair members to surfaces of solid structures.

To be associated with a SERS-active particle, a first specific binding pair member is attracted to the SERS-active substrate. In association, the first specific binding pair member is placed on or near the SERS-active substrate by internal force, external force, or thermal drift (e.g. charge attraction, magnetic field, optical pressure, fluidic pressure, or diffusion). No covalent or ionic bonding is necessary for the association. To be associated with a first specific binding pair member, the SERS-active substrate is placed in proximity to the first specific binding pair member, typically at least within 100 nm of the first specific binding pair member. For example, the first specific binding pair member can be adsorbed on the surface of the SERS-active substrate.

Regarding association of a first specific binding pair member with the SERS-active particle or substrate, it will be recognized that covalent attachment of the first specific binding pair member to the SERS-active particle or substrate, such as a metal nanoparticle, is not required in order to generate an enhanced Raman signal by SERS, SERRS or CARS. For example, where the SERS-active particle or substrate is a metal particle, the first specific binding pair member can be associated with the metal particle by adsorbing the first specific binding pair member to the metal particle. As illustrated in FIG. 2, for example, the metal particles can be negatively charged on the surface due to the distribution of free electrons, and can adsorb to the positively charged part of the first specific binding pair member or a label associated with the first specific binding pair member. Generally, metal particles are mixed in the presence of specific binding pair members to adsorb the first specific binding pair members to the metal particles.

In an illustrative example of a method for adsorbing an antibody to a metal particle, a silver colloid solution is mixed with first specific binding pair members. The silver colloid solution can be made by a known recipe (P. C. Lee and D. Meisel, J. Phys. Chem. 86, 3391 (1992)). To prevent strong binding between silver particles and the antibody, 100 μL of PEG-400 (polyethylene-glycol-400) can be added to the silver solution, followed by incubation at room temperature for 1 hour.

A non-limiting, illustrative example of a method of the present invention is provided in the following paragraph. Antibodies are immobilized by known methods. For example, Xenobind™ Aldehyde slides (Polysciences, Inc., PA, USA) can be used as substrates for methods disclosed herein; before being used, wells on a slide can be prepared by overlaying a piece of cured PDMS of 1 mm thick. The PDMS can have holes of 5 mm in diameter. Antibody (9 ug/mL) can be prepared in 0.33×PBS. Fifty microliters of the antibody can be added to a well on the slide and the slide can be incubated in a humidity chamber at 37° C. for 2 hours. After removing the antibody solution, 50 μL of 1% BSA in a 10 mM glycine solution can be added to each well to quench the aldehyde groups. The slide can then be incubated at 37° C. for another 1 hour, and the wells can be washed 4 times, each with 50 μL PBST washing solution (1×PBS, supplemented with 0.05% Tween-20). To associate silver particles with antibodies, 50 μL of PEG-treated silver colloid solution can be spotted onto the wells; The solution can be incubated at room temperature for another 5 min and the excess solution can optionally be removed. The wells can then be used for protein binding using conditions similar to standard immunoassays, followed by detection of the antibody-metal particle conjugates using SERS. A sample suspected of including a target protein can be applied to each well and incubated at 37° C. for another 1 hour. The wells can be washed four times, each with 50 μL of buffer solution, followed by washing with 50 μL of DI-water once. Finally, 30 μL of DI-water can be added to each well before Raman signal detection of the immobilized antibodies.

Adsorption is a relatively weak chemical binding. Thus, adsorbed particles can be released when the chemical and physical conditions change or when external force is applied. This is utilized in certain aspects of the methods provided herein in order to detect binding of a first specific binding pair member to a second specific binding pair member by a decreased SERS signal.

Adsorption of the first specific binding pair member can be enhanced by the introduction of certain chemical salts, which can also induce aggregation of the metal particles, which typically leads to a stronger SERS signal, as disclosed herein. Therefore, in certain aspects, the first specific binding pair member is contacted with the metal particle in the presence of chemical salts. For example, the first specific binding pair member can be contacted with both an alkali-metal halide salt, such as lithium chloride, and the silver nanoparticles, for example. Lithium chloride can be used, for example, at a concentration of about 50 to about 150 micromolar. Other chemical salts that can be used in the methods provided herein, include sodium chloride, sodium bromide, and sodium iodide.

In certain aspects of the invention, the metal nanoparticles can be covalently attached to first specific binding pair members. Typically binding of the second specific binding pair member to the first specific binding pair member results in a change in the SERS signature rather than inhibition of the SERS signal. The specific binding pair members can be directly attached to the nanoparticles, or can be attached to linker compounds that are covalently or non-covalently bonded to the nanoparticles. Rather than cross-linking two or more nanoparticles together the linker compounds may be used to attach a first specific binding pair member to a nanoparticle or a nanoparticle aggregate. The nanoparticles can be coated with derivatized silanes. Such modified silanes can be covalently attached to first specific binding pair members using standard methods.

Various methods known for cross-linking specific binding pair members to surfaces discussed below can also be used to attach the first specific binding pair member to nanoparticles. It is contemplated that the linker compounds used to attach a first specific binding pair member to can be of almost any length less than about 100 nm.

As indicated above, in certain aspects of the invention, the first specific binding pair member is immobilized on an immobilization substrate. The type of substrate to be used for immobilization of the first specific binding pair member is not limiting as long as it is effective for immobilizing an specific binding pair member while providing access of the first specific binding pair member to the second specific binding pair member and does not inhibit SERS emissions from the first specific binding pair member. The immobilization surface can be magnetic beads, non-magnetic beads, a planar surface, a pointed surface, or any other conformation of solid surface that includes almost any material, so long as the material is sufficiently durable and inert to allow the nucleic acid sequencing reaction to occur. Non-limiting examples of surfaces that can be used include glass, silica, silicate, PDMS, nitrocellulose, nylon, activated quartz, activated glass, polyvinylidene difluoride (PVDF), polystyrene, polyacrylamide, other polymers such as poly(vinyl chloride), poly(methyl methacrylate) or poly(dimethyl siloxane), and photopolymers which contain photoreactive species such as nitrenes, carbenes and ketyl radicals. In certain aspects, the surface can include silver or other metal coated surfaces.

Various methods of attaching specific binding pair members to surfaces are known in the art and can be employed. For example cross-linking agents can be used. Furthermore, functional groups can be covalently attached to cross-linking agents so that binding interactions between specific binding pair members can occur without steric hindrance. Typical cross-linking groups include ethylene glycol oligomers and diamines. Attachment can be by either covalent or non-covalent binding. The cross-linking groups for attaching specific binding pair members to immobilization surfaces are referred to herein as immobilization groups.

As another specific example, immobilization can be achieved by coating a surface with streptavidin or avidin and the subsequent attachment of a biotinylated first specific binding pair member, such as a biotinylated antibody (See Holmstrom et al., Anal. Biochem. 209:278-283, 1993 for method using nucleic acids). Immobilization can also involve coating a silicon, glass or other surface with poly-L-Lys (lysine). Amine residues can be introduced onto a surface through the use of aminosilane for cross-linking.

The first specific binding pair member can be bound to glass by first silanizing the glass surface, then activating with carbodiimide or glutaraldehyde. Alternative procedures can use reagents such as 3-glycidoxypropyltrimethoxysilane (GOP) or aminopropyltrimethoxysilane (APTS). Certain specific binding pair members can be bound directly to membrane surfaces using ultraviolet radiation.

Bifunctional cross-linking reagents can be of use for attaching an specific binding pair member to a surface. The bifunctional cross-linking reagents can be divided according to the specificity of their functional groups, e.g., amino, guanidino, indole, or carboxyl specific groups. Of these, reagents directed to free amino groups are popular because of their commercial availability, ease of synthesis and the mild reaction conditions under which they can be applied. Exemplary methods for cross-linking molecules are disclosed in U.S. Pat. Nos. 5,603,872 and 5,401,511. Cross-linking reagents include glutaraldehyde (GAD), bifunctional oxirane (OXR), ethylene glycol diglycidyl ether (EGDE), and carbodiimides, such as 1-ethyl-3-(3-dimethylaminopropyl) carbodiimide (EDC).

As indicated herein, in certain aspects of the methods provided herein, the first specific binding pair member is associated with a SERS-active particle or substrate by immobilizing the first specific binding pair member on a SERS-active substrate. In these embodiments, the immobilization substrate can also be the SERS-active substrate. Many different types of SERS-active substrates are known in the art. For example, immobilization substrates described above, that include a Raman-active metal, can be used.

In certain examples of this aspect of the methods provided herein, the SERS-active substrate to which a first specific binding pair member is associated and typically immobilized, is a porous metal substrate, such as a porous silicon substrate that includes impregnated metals. Methods are known for coating porous substrates with a uniform layer of one or more metals, such as Raman active metals. Although in particular embodiments of the invention the porous substrates disclosed herein are porous silicon substrates, those embodiments are not limiting. Any porous substrate that is resistant to the application of heat may be used in the disclosed methods, systems and/or apparatus. In certain embodiments, application of heat to about 300° C., 400° C., 500° C., 600° C., 700° C., 800° C., 900° C. or 1,000° C. is contemplated. In some embodiments of the invention, the porous substrate may be rigid. A variety of porous substrates are known, including but not limited to porous silicon, porous polysilicon, porous metal grids and porous aluminum. Exemplary methods of making porous substrates are disclosed in further detail below.

Porous polysilicon substrates can be made by known techniques (e.g., U.S. Pat. Nos. 6,249,080 and 6,478,974). For example, a layer of porous polysilicon can be formed on top of a semiconductor substrate by the use of low pressure chemical vapor deposition (LPCVD). The LPCVD conditions may include, for example, a pressure of about 20 pascal, a temperature of about 640° C. and a silane gas flow of about 600 sccm (standard cubic centimeters) (U.S. Pat. No. 6,249,080). A polysilicon layer may be etched, for example using electrochemical anodization with HF (hydrofluoric acid) or chemical etching with nitric acid and hydrofluoric acid, to make it porous (U.S. Pat. No. 6,478,974). Typically, porous polysilicon layers formed by such techniques are limited in thickness to about 1 μm (micrometer) or less. In contrast, porous silicon can be etched throughout the thickness of the bulk silicon wafer, which has a typical thickness of about 500 μm.

The porous substrates may include one or more layers of nanocrystalline silicon. Various methods for producing nanocrystalline silicon are known (e.g., Petrova-Koch et al., “Rapid-thermal-oxidized porous silicon—the superior photoluminescent Si,” Appl. Phys. Lett. 61:943, 1992; Edelberg, et al., “Visible luminescence from nanocrystalline silicon films produced by plasma enhanced chemical vapor deposition,” Appl. Phys. Lett., 68:1415-1417, 1996; Schoenfeld, et al., “Formation of Si quantum dots in nanocrystalline silicon,” Proc. 7th Int. Conf. on Modulated Semiconductor Structures, Madrid, pp. 605-608, 1995; Zhao, et al., “Nanocrystalline Si: a material constructed by Si quantum dots,” 1st Int. Conf. on Low Dimensional Structures and Devices, Singapore, pp. 467-471, 1995; Lutzen et al., Structural characteristics of ultrathin nanocrystalline silicon films formed by annealing amorphous silicon, J. Vac. Sci. Technology B 16:2802-05, 1998; U.S. Pat. Nos. 5,770,022; 5,994,164; 6,268,041; 6,294,442; 6,300,193). The methods, systems and apparatus disclosed herein are not limited by the method of producing nanocrystalline silicon substrates and any known method may be used.

In aspects of the invention wherein the SERS-active substrate is a metal-coated porous substrate, the substrate is not limited to pure silicon, but may also comprise silicon nitride, silicon oxide, silicon dioxide, germanium and/or other materials known for chip manufacture. Other minor amounts of material may also be present, such as dopants. Porous silicon 110,210 has a large surface area of up to 783 m²/cm³, providing a very large surface for applications such as surface enhanced Raman spectroscopy techniques.

Metals impregnated into porous silicon substrates are typically Raman active metals. Exemplary Raman active metals include, but are not limited to gold, silver, platinum, copper and aluminum. Known methods of metal coating include electroplating; cathodic electromigration; evaporation and sputtering of metals; using seed crystals to catalyze plating (i.e., using a copper/nickel seed to plate gold); ion implantation; diffusion; or any other method known in the art for plating thin metal layers on porous substrates. (See, e.g., Lopez and Fauchet, “Erbium emission from porous silicon one-dimensional photonic band gap structures,” Appl. Phys. Lett. 77:3704-6, 2000; U.S. Pat. Nos. 5,561,304; 6,171,945; 6,359,276.) Another non-limiting example of metal coating includes electroless plating (e.g., Gole et al., “Patterned metallization of porous silicon from electroless solution for direct electrical contact,” J. Electrochem. Soc. 147:3785, 2000). The composition and/or thickness of the metal layer may be controlled to optimize optical and/or electrical characteristics of the metal-coated porous substrates.

Certain methods of the invention provided herein can involve incorporating a label into the specific binding pair members, to enhance their ability to produce a detectable Raman signature. A Raman label can be any organic or inorganic molecule, atom, complex or structure capable of producing a detectable Raman signal, including but not limited to synthetic molecules, dyes, naturally occurring pigments such as phycoerythrin, organic nanostructures such as C₆₀, buckyballs and carbon nanotubes or nanoprisms and nano-scale semiconductors such as quantum dots. Numerous examples of Raman labels are disclosed below. The skilled artisan will realize that such examples are not limiting, and that a Raman label can encompasses any organic or inorganic atom, molecule, compound or structure known in the art that can be detected by Raman spectroscopy.

Non-limiting examples of labels that can be used for Raman spectroscopy include TRIT (tetramethyl rhodamine isothiol), NBD (7-nitrobenz-2-oxa-1,3-diazole), Texas Red dye, phthalic acid, terephthalic acid, isophthalic acid, cresyl fast violet, cresyl blue violet, brilliant cresyl blue, para-aminobenzoic acid, erythrosine, biotin, digoxigenin, 5-carboxy-4′,5′-dichloro-2′,7′-dimethoxy fluorescein, 5-carboxy-2′,4′,5′,7′-tetrachlorofluorescein, 5-carboxyfluorescein, 5-carboxy rhodamine, 6-carboxyrhodamine, 6-carboxytetramethyl amino phthalocyanines, azomethines, cyanines, xanthines, succinylfluoresceins and aminoacridine. Polycyclic aromatic compounds in general can function as Raman labels, as is known in the art. These and other Raman labels can be obtained from commercial sources (e.g., Molecular Probes, Eugene, Oreg.).

Other labels that can be of use include cyanide, thiol, chlorine, bromine, methyl, phosphorus and sulfur. Carbon nanotubes can also be of use as Raman labels. The use of labels in Raman spectroscopy is known (e.g., U.S. Pat. Nos. 5,306,403 and 6,174,677). The skilled artisan will realize that Raman labels should generate distinguishable Raman spectra when bound to different types of nucleotide.

Labels can be attached directly to the specific binding pair members or can be attached via various linker compounds. Raman labels that contain reactive groups designed to covalently react with other molecules, are commercially available (e.g., Molecular Probes, Eugene, Oreg.).

In another embodiment, an apparatus is provided that includes a reaction chamber to contain the specific binding pair member immobilized on a substrate and associated with a SERS-active particle or substrate, a channel in fluid communication with the reaction chamber, and a Raman detection unit operably coupled to the channel, is provided. The apparatus can be used to perform methods provided herein, wherein binding of the first specific binding pair member to the second specific binding pair member is detected by the use of the Raman detection unit.

Microfluidics and nanofluidics can be used to perform methods disclosed herein. In these embodiments, the dimensions of a reaction chamber in which various steps of the reaction are performed, in at least one dimension are in the range of 7 nanometer to 100 millimeters. In general, these embodiments decrease the necessary incubation times over larger reaction chambers. In certain aspects, the reaction chamber is 100 micrometers or less, including, for example, 100 micrometer, 50 micrometer, 25 micrometer, 20 micrometer, 15 micrometer, 10 micrometer, 5 micrometer, 1 micrometer, 500 nm, 250 nm, 100 nm, 50 mm, 25 mm, 20 mm, 15 mm, 10 mm, 9 rim, 8 mm, or 7 nm in at least one dimension.

In some embodiments of the invention, a method disclosed herein can be performed in a micro-electro-mechanical system (MEMS). MEMS are integrated systems that include mechanical elements, sensors, actuators, and electronics. All of those components can be manufactured by known microfabrication techniques on a common chip, including a silicon-based or equivalent substrate (e.g., Voldman et al., Ann. Rev. Biomed. Eng. 1:401-425, 1999). The sensor components of MEMS can be used to measure mechanical, thermal, biological, chemical, optical and/or magnetic phenomena. The electronics can process the information from the sensors and control actuator components such pumps, valves, heaters, coolers, filters, etc. thereby controlling the function of the MEMS.

The electronic components of MEMS can be fabricated using integrated circuit (IC) processes (e.g., CMOS, Bipolar, or BICMOS processes). They can be patterned using photolithographic and etching methods known for computer chip manufacture. The micromechanical components can be fabricated using compatible “micromachining” processes that selectively etch away parts of the silicon wafer or add new structural layers to form the mechanical and/or electromechanical components.

Basic techniques in MEMS manufacture include depositing thin films of material on a substrate, applying a patterned mask on top of the films by photolithographic imaging or other known lithographic methods, and selectively etching the films. A thin film can have a thickness in the range of a few nanometers to 100 micrometers. Deposition techniques of use may include chemical procedures such as chemical vapor deposition (CVD), electrodeposition, epitaxy and thermal oxidation and physical procedures like physical vapor deposition (PVD) and casting.

In some embodiments of the invention, SERS-active particles or substrates, and/or immobilization surfaces are connected to various fluid filled compartments, such as microfluidic channels, nanochannels and/or microchannels. These and other components of the apparatus can formed as a single unit, for example in the form of a chip as known in semiconductor chips and/or microcapillary or microfluidic chips. Alternatively, an immobilization substrate, such as a metal coated porous silicon substrate, can be removed from a silicon wafer and attached to other components of an apparatus. Any materials known for use in such chips may be used in the disclosed apparatus, including silicon, silicon dioxide, silicon nitride, polydimethyl siloxane (PDMS), polymethylmethacrylate (PMMA), plastic, glass, quartz.

Techniques for batch fabrication of chips are well known in the fields of computer chip manufacture and/or microcapillary chip manufacture. Such chips may be manufactured by any method known in the art, such as by photolithography and etching, laser ablation, injection molding, casting, molecular beam epitaxy, dip-pen nanolithography, chemical vapor deposition (CVD) fabrication, electron beam or focused ion beam technology or imprinting techniques. Non-limiting examples include conventional molding with a flowable, optically clear material such as plastic or glass; photolithography and dry etching of silicon dioxide; electron beam lithography using polymethylmethacrylate resist to pattern an aluminum mask on a silicon dioxide 120 substrate, followed by reactive ion etching. Known methods for manufacture of nanoelectromechanical systems may be used for certain embodiments of the invention. (See, e.g., Craighead, Science 290: 1532-36, 2000.) Various forms of microfabricated chips are commercially available from, e.g., Caliper Technologies Inc. (Mountain View, Calif.) and ACLARA BioSciences Inc. (Mountain View, Calif.).

In certain embodiments of the invention, part or all of the apparatus can be selected to be transparent to electromagnetic radiation at the excitation and emission frequencies used for Raman spectroscopy, such as glass, silicon, quartz or any other optically clear material. For fluid-filled compartments that may be exposed to various biomolecules, such as proteins, peptides, nucleic acids, nucleotides and the like, the surfaces exposed to such molecules may be modified by coating, for example to transform a surface from a hydrophobic to a hydrophilic surface and/or to decrease adsorption of molecules to a surface. Surface modification of common chip materials such as glass, silicon, quartz and/or PDMS is known in the art (e.g., U.S. Pat. No. 6,263,286). Such modifications may include, but are not limited to, coating with commercially available capillary coatings (Supelco, Bellafonte, Pa.), silanes with various functional groups such as polyethyleneoxide or acrylamide, or any other coating known in the art.

The first specific binding pair member is detected in the methods provided herein, by SERS using a Raman detection unit. The Raman detection unit includes a laser excitation and a wavelength selective detector. The light source is typically a laser light, as known in the art and discussed in more detail herein. Light from the light source is projected at the first specific binding pair member and detected by the detector.

The detection unit includes an excitation source, such as a laser, and a Raman spectroscopy detector. The excitation source illuminates the reaction chamber or channel with an excitation beam. The excitation beam interacts with the first specific binding pair member, resulting in the excitation of electrons to a higher energy state. As the electrons return to a lower energy state, they emit a Raman emission signal that is detected by the Raman detector.

Data can be collected from a detector, such as a spectrometer or a monochromator array and provided to an information processing and control system. The information processing and control system can perform standard procedures known in the art, such as subtraction of background signals. Furthermore, the information processing and control system can analyze the data to determine whether a change in the SERS signal has occurred between SERS spectra obtained before versus after contacting the first specific binding pair member with the second specific binding pair member. For example, the information processing and control system can use standard statistical methods.

As indicated above, an apparatus for performing the methods provided herein, typically includes a reaction chamber. A reaction chamber can be designed to hold an immobilization surface, first specific binding pair member, second specific binding pair member, and/or a Raman-active particle or surface in an aqueous environment. The reaction chamber can be designed to be temperature controlled, for example by incorporation of Pelletier elements or other methods known in the art. Methods of controlling temperature for low volume liquids used in nucleic acid polymerization are known in the art. (See, e.g., U.S. Pat. Nos. 5,038,853, 5,919,622, 6,054,263 and 6,180,372.)

The reaction chamber and any associated fluid channels can provide connections to a detection unit, to a waste port, to a loading port, to a source of metal particles, or to an specific binding pair member. The reaction chamber can be manufactured in a batch fabrication process, as known in the fields of computer chip manufacture or microcapillary chip manufacture.

The reaction chamber and other components of the apparatus can be manufactured as a single integrated chip. Such a chip can be manufactured by methods known in the art: such as by photolithography and etching, laser ablation, injection molding, casting, molecular beam epitaxy, dip-pen nanolithography, chemical vapor deposition (CVD) fabrication, electron beam or focused ion beam technology or imprinting techniques. Non-limiting examples include conventional molding with a flowable, optically clear material such as plastic or glass; photolithography and dry etching of silicon dioxide; electron beam lithography using polymethylmethacrylate resist to pattern an aluminum mask on a silicon dioxide substrate, followed by reactive ion etching. Microfluidic channels can be made by molding polydimethylsiloxane (PDMS) according to Anderson et al. (“Fabrication of topologically complex three-dimensional microfluidic systems in PDMS by rapid prototyping,” Anal. Chem. 72:3158-3164, 2000). Methods for manufacture of nanoelectromechanical systems can be used. (See, e.g., Craighead, Science 290:1532-36, 2000.) Microfabricated chips are commercially available from sources such as Caliper Technologies Inc. (Mountain View, Calif.) and ACLARA BioSciences Inc. (Mountain View, Calif.).

Any materials known for use in integrated chips can be used in an apparatus to perform methods provided herein, including silicon, silicon dioxide, silicon nitride, polydimethyl siloxane (PDMS), polymethylmethacrylate (PMMA), plastic, glass, quartz, etc. Part or all of the apparatus can be selected to be transparent to electromagnetic radiation at the excitation and emission frequencies used for Raman spectroscopy, such as glass, silicon, quartz or any other optically clear material. For fluid-filled compartments that can be exposed to nucleic acids and/or nucleotides, such as the reaction chamber, microfluidic channel, nanochannel or microchannel, the surfaces exposed to such molecules can be modified by coating, for example to transform a surface from a hydrophobic to a hydrophilic surface and/or to decrease adsorption of molecules to a surface. Surface modification of common chip materials such as glass, silicon and/or quartz is known in the art (e.g., U.S. Pat. No. 6,263,286). Such modifications can include, but are not limited to, coating with commercially available capillary coatings (Supelco, Bellafonte, Pa.), silanes with various functional groups such as polyethyleneoxide or acrylamide, or any other coating known in the art.

Specific binding pair members can be moved down a microfluidic channel, nanochannel or microchannel to the reaction chamber and to the detection unit. A microchannel or nanochannel can have a diameter between about 3 nm and about 1 μm. The diameter of the channel can be selected to be slightly smaller in size than an excitatory laser beam. The channel can include a microcapillary (available, e.g., from ACLARA BioSciences Inc., Mountain View, Calif.) or a liquid integrated circuit (e.g., Caliper Technologies Inc., Mountain View, Calif.). Such microfluidic platforms require only nanoliter volumes of sample. Nucleotides can move down a microfluidic channel by bulk flow of solvent, by electro-osmosis or by any other technique known in the art.

Micro fabrication of microfluidic devices, including microcapillary electrophoretic devices has been discussed in, e.g., Jacobsen et al. (Anal. Biochem, 209:278-283,1994); Effenhauser et al. (Anal. Chem. 66:2949-2953, 1994); Harrison et al. (Science 261:895-897, 1993) and U.S. Pat. No. 5,904,824. Typically, these methods include photolithographic etching of micron scale channels on silica, silicon or other crystalline substrates or chips, and can be readily adapted for use in the disclosed methods and apparatus. Smaller diameter channels, such as nanochannels, can be prepared by known methods, such as coating the inside of a microchannel to narrow the diameter, or using nanolithography, focused electron beam, focused ion beam or focused atom laser techniques.

A detection unit can be designed to obtain Raman signals generated by specific binding pair members. This typically involves SERS detection. Variations on surface enhanced Raman spectroscopy (SERS) or surface enhanced resonance Raman spectroscopy (SERRS) can be used. In SERS and SERRS, the sensitivity of the Raman detection is enhanced by a factor of 10⁶ or more for molecules adsorbed on, or otherwise associated with, roughened metal surfaces, such as silver, gold, platinum, copper or aluminum surfaces, or on nanostructured surfaces.

A non-limiting example of a detection unit is disclosed in U.S. Pat. No. 6,002,471. In this embodiment, the excitation beam is generated by either a frequency doubled Nd:YAG laser at 532 nm wavelength or a frequency doubled Ti:sapphire laser at 365 nm wavelength. However, the excitation wavelength can vary considerably, without limiting the methods provided herein. Pulsed laser beams or continuous laser beams can be used. The excitation beam passes through confocal optics and a microscope objective, and is focused onto the reaction chamber. The Raman emission light from the specific binding pair members is collected by the microscope objective and the confocal optics and is coupled to a monochromator for spectral dissociation. The confocal optics includes a combination of dichroic filters, barrier filters, confocal pinholes, lenses, and mirrors for reducing the background signal. Standard full field optics can be used as well as confocal optics. The Raman emission signal is detected by a Raman detector. The detector includes an avalanche photodiode interfaced with a computer for counting and digitization of the signal.

Alternative embodiments of detection units are disclosed, for example, in U.S. Pat. No. 5,306,403, including a Spex Model 1403 double-grating spectrophotometer equipped with a gallium-arsenide photomultiplier tube (RCA Model C31034 or Burle Industries Model C3103402) operated in the single-photon counting mode. The excitation source is a 514.5 nm line argon-ion laser from SpectraPhysics, Model 166, and a 647.1 nm line of a krypton-ion laser (Innova 70, Coherent).

Alternative excitation sources include a nitrogen laser (Laser Science Inc.) at 337 nm and a helium-cadmium laser (Liconox) at 325 nm (U.S. Pat. No. 6,174,677). The excitation beam can be spectrally purified with a bandpass filter (Corion) and can be focused on the reaction chamber using a 6× objective lens (Newport, Model L6X). The objective lens can be used to both excite the nucleotides and to collect the Raman signal, by using a holographic beam splitter (Kaiser Optical Systems, Inc., Model HB 647-26N18) to produce a right-angle geometry for the excitation beam and the emitted Raman signal. A holographic notch filter (Kaiser Optical Systems, Inc.) can be used to reduce Rayleigh scattered radiation. Alternative Raman detectors include an ISA HR-320 spectrograph equipped with a red-enhanced intensified charge-coupled device (RE-ICCD) detection system (Princeton Instruments). Other types of detectors can be used, such as charged injection devices, photodiode arrays or phototransistor arrays.

Any suitable form or configuration of Raman spectroscopy or related techniques known in the art can be used for detection of specific binding pair members, including but not limited to normal Raman scattering, resonance Raman scattering, surface enhanced Raman scattering, surface enhanced resonance Raman scattering, coherent anti-Stokes Raman spectroscopy (CARS), stimulated Raman scattering, inverse Raman spectroscopy, stimulated gain Raman spectroscopy, hyper-Raman scattering, molecular optical laser examiner (MOLE) or Raman microprobe or Raman microscopy or confocal Raman microspectrometry, three-dimensional or scanning Raman, Raman saturation spectroscopy, time resolved resonance Raman, Raman decoupling spectroscopy or UV-Raman microscopy.

In certain aspects, the methods provided herein include detecting the fist specific binding pair member using CARS. After associating a first specific binding pair member with a SERS particle or substrate, the first specific binding pair member can be detected using SECARS detection. As is known, CARS detects coherent anti-Stokes Raman scattering, which is the non-linear optical analogue of spontaneous Raman scattering. In this technique a particular Raman transition is coherently driven by two laser fields—the so-called “Pump laser” and “Stokes laser,” generating an anti-Stokes signal field (Müller et al., CARS microscopy with folded BoxCARS phasematching. J. Microsc. 197:150-158, 2000). The coherent nature of the process permits efficient coupling of the laser fields to a particular vibrational mode, increasing the signal from this mode by many orders of magnitude. SECARS is CARS detection of a molecule associated with a SERS substrate.

As indicated above, in another embodiment, provided herein is a kit incorporating a specific binding pair, such as an antibody, as well as SERS particle or substrate. In certain aspects, the kit can include an specific binding pair member associated with the SERS particle or substrate. Furthermore, the kit can include an immobilization substrate. In certain aspects, the first specific binding pair member can be included in the kit attached to the immobilization substrate, and optionally associated with the SERS particle or substrate. The kit also can contain, for example, reagents for labeling a specific binding pair member.

The following examples are intended to illustrate but not limit the invention.

### EXAMPLE 1

### SERS Detection of Unlabeled Antibody

This example illustrates the detection of an antibody using SERS. Antibody molecules were immobilized on the gold-coated substrate by using EDC chemistry (1-Ethyl-3-[3-dimethylaminopropyl]carbodiimide Hydrochloride), developed by and available from Pierce (Rockford, Ill.). The control was a blank substrate with EDC treatment and no antibody. Eighty microliters of a mixture of colloidal silver (synthesized by the recipe published by Lee and Meisel, J. Phys. Chem. 1982, 986, 3391-3396) and lithium chloride salt solution were applied onto the sample and the control before spectrum collection.

A Raman microscope was used to collect the spectrum from the antibody sample and the control sample. The Raman microscope included an argon ion laser (Coherent, Santa Clara, Calif.), an optical microscope (Nikon), optical filters (Kaiser Optical, Ann Arbor, Mich.), a spectrograph (Acton Research, Acton, Mass.), and a CCD camera (Roper Scientific, Princeton, N.J.). The laser provided less than 100 mW at the focus, and each spectrum was collected for 100 milliseconds.

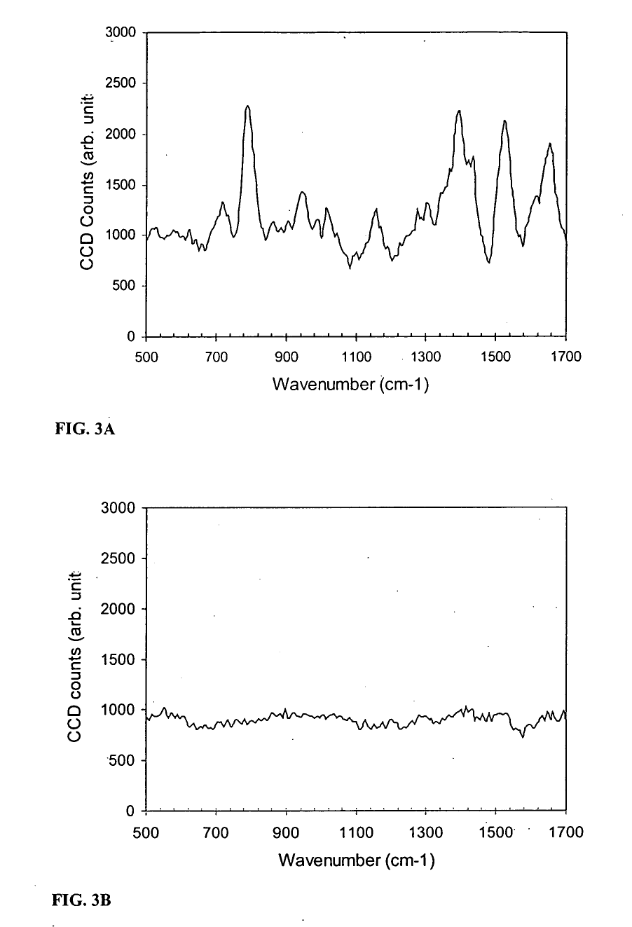

As illustrated in FIGS. 3A and 3B, the antibody sample produced a detectable SERS spectrum that was not present in the control sample. These results demonstrate that antibodies generate a SERS signal.

Although the invention has been described with reference to the above example, it will be understood that modifications and variations are encompassed within the spirit and scope of the invention. Accordingly, the invention is limited only by the following claims.

1. A method to detect binding of a first specific binding pair member to a second specific binding pair member, comprising:
a) associating a first specific binding pair member with a surface-enhanced Raman scattering-active particle or substrate;
b) contacting the first specific binding pair member associated with the surface-enhanced Raman scattering-active particle or substrate with a second specific binding pair member; and
c) detecting binding of the second specific binding pair member to the first specific binding pair member by detecting a difference in a surface-enhanced Raman scattering signal of the first specific binding pair member before contacting the first specific binding pair member with the second specific binding pair member and after contacting the first specific binding pair member with the second specific binding pair member, thereby detecting binding of the first specific binding pair member to the second specific binding pair member.

2. The method of claim 1, wherein the surface-enhanced Raman scattering-active particle or substrate associated with the first specific binding pair member is a metal particle.

3. The method of claim 2, wherein the first specific binding pair member is associated with the metal particle by adsorbing the first specific binding pair member to the surface-enhanced Raman scattering surface.

4. The method of claim 3, wherein the metal particle comprises colloidal silver or gold.

5. The method of claim 3, wherein the first specific binding pair member is immobilized on an immobilization substrate prior to associating with the surface-enhanced Raman scattering-active surface.

6. The method of claim 1, wherein the difference in the surface-enhanced Raman scattering signal is a decrease in the signal.

7. The method of claim 6, wherein binding of the second specific binding pair member to the first specific binding pair member dissociates the first specific binding pair member from the metal particle.

8. The method of claim 1, wherein the difference in the surface-enhanced Raman scattering signal is an increase in the signal.

9. The method of claim 3, wherein adsorption is detected before the second specific binding pair member is contacted with the first specific binding pair member.

10. The method of claim 9, wherein adsorption is detected by detecting an increase in a surface-enhanced Raman scattering signal generated by the first specific binding pair member after contacting the first specific binding pair member with the metal particle.

11. The method of claim 3, wherein the first specific binding pair member is associated with the metal particle in the presence of a chemical salt.

12. The method of claim 11, wherein the chemical salt is lithium chloride.

13. The method of claim 1, wherein the first specific binding member is a protein and the second specific binding pair member is a protein.

14. The method of claim 13, wherein the first or second specific binding pair member is an antibody molecule, or fragment thereof.

15. The method of claim 1, wherein the first specific binding pair member is a receptor and the second specific binding pair member is a ligand.

16. The method of claim 1, wherein the first or second specific binding pair member is a nucleic acid molecule and the other of the first or second specific binding pair member is a protein.

17. The method of claim 13, wherein the first specific binding pair member is bound to a surface-enhanced Raman scattering label.

18. The method of claim 17, wherein the surface-enhanced Raman scattering label is deoxy-adenosine monophosphate.

19. The method of claim 18, wherein surface enhanced coherent anti-Stokes Raman spectroscopy is used to detect the first specific binding pair member.

20. The method of claim 1, wherein the first specific binding pair member is associated with the surface-enhanced Raman scattering-active particle or substrate by immobilizing the first specific binding pair member on a surface-enhanced Raman scattering-active substrate.

21. The method of claim 20, wherein the surface-enhanced Raman scattering-active substrate comprises a porous silicon substrate comprising impregnated metals.

22. A method to detect binding of an antibody, or fragment thereof, to an antigen, comprising:
a) immobilizing an antibody on an immobilization substrate;
b) contacting the immobilized antibody with a metal particle to adsorb the immobilized antibody on the metal particle;
c) contacting the immobilized antibody with an antigen; and
d) detecting binding of the antigen to the antibody, or fragment thereof, by detecting a difference in a surface-enhanced Raman scattering signal generated by the antibody before contacting the antibody with the antigen and after contacting the antibody with the antigen, thereby detecting binding of the antibody to the antigen.

23. The method of claim 22, wherein the antibody, or fragment thereof, is a whole antibody molecule.

24. The method of claim 22, wherein the antibody, or fragment thereof, is a Fab fragment.

25. The method of claim 22, wherein the metal particle comprises colloidal gold or silver.

26. A method to detect an analyte in a biological sample, comprising:
a) immobilizing a first specific binding pair member on a surface, wherein the first specific binding pair member binds the analyte;
b) contacting the immobilized first specific binding pair member with a metal particle to adsorb the immobilized first specific binding pair member on the metal particle;
c) contacting the immobilized first specific binding pair member adsorbed on the metal particle with the biological sample; and
d) detecting a surface-enhanced Raman scattering signal generated by the immobilized first specific binding pair member before contacting the immobilized first specific binding pair member with the second specific binding pair member and after contacting the first specific binding pair member with the second specific binding pair member, wherein a difference in the detected surface-enhanced Raman scattering signals is indicative of the presence of the analyte in the biological sample.

27. The method of claim 26, wherein the first specific binding pair member is an antibody, or fragment thereof.

28. The method of claim 26, wherein the metal particle comprises colloidal gold or silver.

29. The method of claim 26, wherein the first specific binding pair member is adsorbed on the metal particle in the presence of lithium chloride.

30. The method of claim 26, wherein the biologic sample comprises serum.

31. A method to detect an antibody or a fragment thereof, comprising:
a) immobilizing the antibody, or fragment thereof, on a surface;
b) contacting the antibody, or fragment thereof, with a metal particle to adsorb the immobilized antibody on the metal particle; and
c) detecting a surface-enhanced Raman scattering signal of the immobilized antibody, or fragment thereof, thereby detecting the antibody, or fragment thereof.

32. The method of claim 30, wherein the antibody, or fragment thereof, is a whole antibody molecule.

33. The method of claim 30, wherein the antibody, or fragment thereof, is a Fab fragment.

34. The method of claim 30, wherein the metal particle comprises colloidal gold or silver.
